(19) United States
(12) Patent Application Publication
(10) Pub. No.: US 2005/0026205 A1
(43) Pub. Date: Feb. 3, 2005

(54) Method of polishing metal and metal/dielectric structures

(76) Inventors: Lothar Puppe, Burscheid (DE); Gerd Passing, Koln (DE); Kristina Vogt, Lohmar (DE); Valentina Terzieva, Heverlee (BE)

Correspondence Address: BAYER MATERIAL SCIENCE LLC, 100 BAYER ROAD, PITTSBURGH, PA, 15205, US

(21) Appl. No.: 10/929,227

(22) Filed: Aug. 30, 2004

Related U.S. Application Data
(62) Division of application No. 10/278,639, filed on Oct. 23, 2002, now abandoned.

(30) Foreign Application Priority Data
Oct. 26, 2001 (DE) 10152993.7

(51) Int. Cl. C12Q 1/68

(52) U.S. Cl. 435/6

(57) ABSTRACT

A composition for the chemical-mechanical polishing of metal and metal/dielectric structures, containing 7 to 100% by volume of a cationically stablilized silica sol which contains 30% by weight of SiO₂ and the SiO₂ particles of which have a mean particle size of less than 300 nm, with a pH of from 4 to 10, is distinguished by a TaN removal rate of ≧40 nm per min and an improved barrier layer:metal selectivity of at least 2:1 or greater and a barrier layer:dielectric selectivity of at least 2:1 or above.

### FIELD OF THE INVENTION

The present invention relates to a composition for the chemical-mechanical polishing (CMP) of metal and metal/dielectric structures, to a process for its production and to its use.

### BRIEF DESCRIPTION OF THE PRIOR ART

Integrated semiconductor circuits (ICs) comprise structured semiconducting, nonconductive and electrically conductive thin films. These structured films are usually produced by a film material being applied by vapour deposition, for example, and structured by means of a microlithographic process. The combination of the various semiconducting, nonconductive and conductive layer materials produces the electronic circuit elements of the IC, such as for example transistors, capacitors, resistors and wiring.

The quality of an IC and its function is crucially dependent on the accuracy with which the various layer materials can be applied and structured. However, as the number of layers increases, the planarity of the layers decreases considerably. Beyond a certain number of layers, this leads to one or more functional elements of the IC failing and therefore to the entire IC failing.

The reduction in the planarity of the layers results from the build-up of new layers when these layers have to be applied to layers which have already been structured. The structuring gives rise to differences in height which may amount to up to 0.6 μm per layer. These differences in height are cumulative from layer to layer and mean that the next layer is no longer applied to a planar surface, but rather to a non-planar surface. A first consequence is that the layer which is subsequently applied has a non-uniform thickness. In extreme cases, flaws and defects are formed in the electronic functional elements and the contacts lack quality. Moreover, uneven surfaces lead to problems with the structuring. To make it possible to produce sufficiently small structures, an extremely high imaging accuracy (DOF, depth of focus) is required in the microlithographic process step. However, these structures can only be sharply focused in one plane; the greater certain locations deviate from this plane, the more blurred the imaging becomes.

To solve this problem, the process known as chemical-mechanical polishing (CMP) is carried out. CMP results in global planarization of the structured surface by removing elevated parts of the layer until a planar layer is obtained. As a result, the next layer can be built up on a planar surface without height differences, and the precision of structuring and the ability of the elements of the IC to function are retained.

A CMP step is carried out with the aid of special polishing machines, polishing pads and polishing abrasives (polishing slurries). A polishing slurry is a composition which, in combination with the polishing pad on the polishing machine, is responsible for removing the material which is to be polished.

A wafer is a polished disc of silicon on which integrated circuits are built up.

An overview of CMP technology is given, for example, in B. L. Mueller, J. S. Steckenrider Chemtech (1998) pp. 38-46.

Particularly in polishing steps in which semiconductor layers are involved, the demands imposed on the accuracy of the polishing step and therefore on the polishing slurry are particularly high.

A range of parameters which are used to characterize the effect of the polishing slurry are used as an assessment scale for the effectiveness of polishing slurries. These parameters include the abrasion rate, i.e. the rate at which the material which is to be polished is removed, the selectivity, i.e. the ratio of the polishing rates of material which is to be polished with respect to further materials which are present, and also variables relating to the uniformity of planarization. Variables used for the uniformity of the planarization are usually the within wafer non-uniformity (WIWNU) and the wafer to wafer non-uniformity (WTWNU), and also the number of defects per unit area.

What is known as the Cu damascene process is increasingly used for the production of integrated circuits (ICs) (cf. for example “Microchip Fabrication: A Practical Guide to Semiconductor Processing”, Peter Van Zant, 4^{th} ed., McGraw-Hill, 2000, pp. 401-403 and 302-309 and “Copper CMP: A Question of Tradeoffs”, Peter Singer, Semiconductor International, Verlag Cahners, May 2000, pp. 73-84). In this case, it is necessary for a Cu layer to undergo chemical-mechanical polishing with a polishing slurry (the so-called Cu-CMP process), in order for the Cu interconnects to be produced. The finished Cu interconnects are embedded in a dielectric. Between Cu and the dielectric there is a barrier layer. The prior art for the Cu-CMP process is a two-step process, i.e. the Cu layer is firstly polished using a polishing slurry which ensures that a large amount of Cu is removed. Then, a second polishing slurry is used in order to produce the final planar surface with the smooth and brightly polished dielectric and the embedded interconnects.

The first polishing step uses a polishing slurry with a high selectivity, i.e. the abrasion rate for Cu is as high as possible and the abrasion rate for the material of the barrier layer below it is as low as possible. The polishing process is stopped automatically as soon as the barrier layer is uncovered below the Cu. Since the complete removal of Cu residues on the barrier layer takes some time (known as “over-polishing”), during this time, at the locations where the embedded Cu interconnects are situated in the dielectric, the Cu of the interconnect continues to be abraded at a considerable rate. This effect is known as dishing. Therefore, depending on the concept and/or quality of the surface obtained in the first polishing step, a polishing slurry which is selective or non-selective with respect to the materials which are to be polished, namely Cu, barrier layer and dielectric, is used for the second polishing step.

If, in the first step, uniform removal of Cu with only small amounts of residual Cu on the barrier layer can be achieved, it is advantageous to use a slurry which is highly selective with respect to the barrier layer for the second step, in order to obtain a uniformly polished surface.

On the other hand, if the first polishing step produces a surface which still contains Cu on the barrier layer, it is recommended to use a polishing slurry which is non-selective with respect to the barrier layer. When a non-selective polishing slurry is being used, i.e. when the removal rate is approximately equal for Cu, barrier layer and dielectric, the entire wafer surface is levelled (planarized) uniformly by the polishing process. In this concept, part of the dielectric layer has to be sacrificed, which represents a drawback on account of the need to deposit relatively thick dielectric and Cu layers. When the non-selective polishing slurry is being used, it is essential for the polishing slurry to have the same planarization efficiency for all three materials which are to be polished. Moreover, the Cu interconnects produced must have a minimum thickness, i.e. there should not be too much material removed from the dielectric layer and the Cu interconnects, which must be monitored during the polishing process.

When a selective polishing slurry is used for the second step, the removal rate for the barrier layer is higher than for the Cu. In this concept, the targeted removal of the barrier layer reduces the phenomenon known as dishing of the Cu interconnects. The loss of dielectric (erosion) and therefore, as a corollary, of the Cu interconnect layer thickness is therefore lower.

Polishing slurries with selective removal rates are already known from the prior art. For example, WO-A-99/64527, Example 3, discloses a polishing slurry based on silica sol containing 2% of H₂O₂ and having a pH of 10.5, which has a Cu:Ta:dielectric (in this case SiO₂, also referred to as oxide) selectivity of 1:1.6:4. However, this known polishing slurry leads to very considerable amounts of the oxide being removed as soon as the barrier layer has been polished away, and therefore to an uneven wafer surface. The phenomenon known as “oxide erosion” is even intensified. The term “oxide erosion” is explained in “Copper CMP: A Question of Tradeoffs”, Peter Singer, Semiconductor International, Verlag Cahners, May 2000, pp. 73-84.

WO-A-00/00567, Example 3, No. 3 has disclosed a polishing slurry with aluminium oxide as abrasive. This results in a Cu:Ta:oxide selectivity ratio of 1:4.5:2, with which it is possible to avoid the oxide erosion; however, a drawback of this polishing slurry is the low removal rate for the barrier layer comprising Ta of 300 Å/min, which slows the production process, and the high hardness of the aluminium oxide, which leads to increased amounts of scratches on the wafer surface.

EP-A 1 069 168 describes, for example for Cu:TaN:SiO₂, selectivities of 1:1.04:0.042. In this case, the amount of SiO₂ removed is too low, leading to dishing at the Cu. Moreover, an agent which makes the removal more intensive is also required.

Furthermore, it is known to add certain additives to the polishing slurry in order to increase the rates at which the metals are removed and/or to adjust the selectivity of the polishing slurry. Oxidizing agents, carboxylic acids and complex-forming agents are known for this purpose. It is known from WO-A-99/64527 and WO-A-99/67056 that silica sols in a basic medium effect high oxide-removal rates, which is the state of the art for pure oxide polishing. WO-A-99/64527 adds polyvinylpyrrolidones (PVPs) to the polishing slurry in order to reduce the oxide-removal rate.

The polishing slurries described above all have the drawback that the selectivities, in particular the Cu:oxide selectivity, have to be adjusted by the addition of, for example, film-forming agents or organic compounds, and the Cu:oxide selectivity, which is predetermined by the abrasive and pH, is unsuitable.

Furthermore, all the known polishing slurries contain H₂O₂ or other oxidizing agents, in order to increase the metal removal rates.

Therefore, the object was set of providing a polishing slurry which is improved compared to the prior art and has a satisfactory removal rate for the barrier layer and a barrier layer:metal selectivity of at least 2:1 or above and a barrier layer:dielectric selectivity of at least 2:1 or above, which can preferably be used without the addition of oxidizing agents.

### SUMMARY OF THE INVENTION

Surprisingly, it has now been found that this object is achieved by a composition which contains a cationically stabilized silica sol with a mean particle diameter of <300 nm as abrasive.

Therefore, the subject matter of the invention is a composition containing 7 to 100% by volume of a cationically stabilized silica sol which contains 30% by weight of SiO₂ and the SiO₂ particles of which have a mean particle size of less than 300 nm, with a pH of from 4 to 10, and less than 0.05% by weight of oxidizing agent.

In this context, the term mean particle size is to be understood as meaning the particle size diameter at d₅₀, as determined using an ultracentrifuge.

To measure the particle sizes in the nanometer range, in addition to electron microscope images, various further methods are also suitable, such as for example laser correlation spectroscopy, ultrasound measurements or measurements using an ultracentrifuge (H. G. Müller, Colloid & Polymer Science 267 (1989) p. 1113). The ultracentrifuge is particularly suitable for forming particle size distributions, on account of its sharpness of separation.

### DETAILED DESCRIPTION OF THE INVENTION

The pH of the composition according to the invention lies in the range from 4 to 10. The range from 5 to 9 is preferred, and the range from 6 to 8 is very particularly preferred. The given pH values are determined at 25° C. The pH of the composition is preferably set by adding a base to the composition. The quantity of base depends on the desired pH. Examples of suitable bases are KOH, NH₄OH, TMAH, guanidine, guanidine carbonate, K₂CO₃ or similar bases which do not contain sodium; the use of potassium hydroxide is preferred. The base is preferably added in the form of an aqueous solution. It is particularly preferred to add an aqueous solution of potassium hydroxide. The composition according to the invention particularly preferably contains 0.001 to 30 g/l of potassium hydroxide (100% strength).

In the context of the present invention, the following definitions of terms apply:

The term metal encompasses, by way of example, the elements W, Al, Cu, Si, Ru, Pt and Ir and/or alloys and carbides thereof.

The term dielectric encompasses, by way of example, organic and inorganic dielectrics. Examples of organic dielectrics are SiLK™ (Dow Chemical Company), polyimides, fluorinated polyimides, diamond-like carbons, polyarylethers, polyarylenes, parylene N, cyclotenes, polynorbornenes and Teflon. Inorganic dielectrics are based, for example, on SiO₂ glass as the principal constituent. Carbon, fluorine, phosphorus and/or boron compounds may be present as additional constituents. Conventional designations for these dielectrics are, for example, FSG, PSG, BSG or BPSG, where SG represents spin-on glass. Various fabrication methods are known for the fabrication of these layers (cf. for example Peter Van Zant, 4^{th} ed., McGraw-Hill, 2000, pp. 363-376 and pp. 389-391). Moreover, silsesquioxanes (HSQ, MSQ) are known as dielectrics which are highly polymerized and are close to the inorganic state.

The term barrier layer encompasses layers of Ta, TaSi, TaN, TaSiN, Ti, TiN, WN, WSiN, SiC, silicon oxynitride, silicon oxycarbide, silicon oxycarbonitride Si₃N₄ and/or silicon oxide, for example.

The use of Ta and TaN as barrier layer is preferred for the fabrication of integrated circuits.

Metals such as Cu, Al or W are preferably used for the fabrication of interconnects for integrated circuits.

SiO₂ and modified SiO₂ glasses are preferably used as dielectrics. Silica sol in the context of the invention is a sol whose colloidal SiO₂ particles are cationically stabilized. The cations are preferably H⁺ and/or K⁺ ions. The primary particles of the silica sol are not aggregated. The mean particle size of the SiO₂ particles in the silica sol which is to be used according to the invention is less than 300 nm. The mean particle size is preferably from 20 to 100 nm, particularly preferably from 30 to 80 nm. The composition according to the invention contains 7 to 100% by volume, preferably 10 to 80% by volume and particularly preferably 17 to 70% by volume of a silica sol which contains 30% by weight of SiO₂, corresponding to an absolute value of 2 to 30% by weight of SiO₂, preferably 3 to 24% by weight of SiO₂, particularly preferably 5 to 21% by weight of SiO₂, based on the composition.

An H⁺-stabilized silica sol has a typical pH of 1.5 to 2.5. At higher pHs, H⁺ is replaced by K⁺, the transition being gradual. A silica sol with a pH of 7 or above is regarded as K⁺-stabilized. The silica sols which are stabilized by H⁺ and/or K⁺ ions are known or can be produced in a manner which is known per se (cf. for example K. K. Iler “The Chemistry of Silica”, Wiley & Sons, New York, 1979, pp. 355-360).

Further customary additives, such as for example anticorrosion agents for the metals, may be added to the composition according to the invention. Examples of suitable anticorrosion agents are benzotriazole, 6-tolyltriazole and phosphates in amounts from 0.0001 to 10% by weight.

Moreover, complex-forming agents for the metals, which make the metals water-soluble, such as for example citric acid or citrates, EDTA, NTA, IDS and amino acids may be added to the composition according to the invention in amounts of from 0.001 to 10% by weight.

The composition according to the invention contains less than 0.05% by weight of oxidizing agents. The composition according to the invention particularly preferably contains 0 to 0.01-% by weight of oxidizing agents. The composition according to the invention is particularly preferably free of oxidizing agents. All conventional oxidizing agents can be considered as oxidizing agents, in particular HNO₃, AgNO₃, CuClO₄, H₂SO₄, H₂O₂, HOCl, KMnO₄, ammonium persulphate, ammonium oxalate, Na₂CrO₄, UHP, iron perchlorate, iron chloride, iron citrate and iron nitrate, HIO₃, KIO₃ and HClO₃.

Furthermore, the invention relates to a process for producing the composition according to the invention, characterized in that a 30 or 40% by weight cationically stabilized silica sol is diluted by the addition of water to a solids content of 7 to 100% by volume, and then a pH of from 4 to 10 is set by addition of a sufficient quantity of base with stirring.

If a silica sol which is stabilized with H⁺ ions is used to produce the composition according to the invention, it can be converted into a K⁺-stabilized silica sol by adding KOH. After KOH has been added, the silica sol is to be stirred until an equilibrium has been established between the cations at the silica sol surface. The KOH is expediently in dissolved form.

The pH of the composition according to the invention is preferably adjusted by adding potassium hydroxide to the silica sol. After potassium hydroxide has been added, the silica sol is stirred until the pH has stabilized. A silica sol with a pH of 1.5 to 2.5 is preferably used to produce compositions with a pH of <6. A silica sol with a pH of 7 or above is preferably used to produce compositions with a pH of >6.

The compositions according to the invention can be used as polishing slurry for the chemical-mechanical polishing of metal and metal/dielectric structures. This novel use likewise forms the subject matter of the present invention. In particular, the composition according to the invention can be used as polishing slurry for the fabrication of semiconductors, integrated circuits and microelectromechanical systems.

The composition according to the invention is preferably used as polishing slurry for the chemical-mechanical polishing of metal and metal/dielectric structures, in particular of integrated circuits and microelectromechanical systems with structures made from metal and dielectrics which are built up on Si wafers.

The metals are preferably W, Al, Cu, Si, Ru, Pt and Ir and/or alloys and carbides thereof.

The dielectrics are preferably SiLK™, polyimides, fluorinated polyimides, diamond-like carbons, polyarylethers, polyarylenes, parylene N, cyclotenes, polynorbonenes, Teflon, silsesquioxanes or SiO₂ glass or mixtures thereof.

The metal/dielectric structures are preferably structures comprising Cu/SiO₂.

The barrier layer is preferably Ta or TaN.

The compositions according to the invention are distinguished by a barrier layer removal rate, in particular a TaN removal rate, of ≧40 nm per min and a barrier layer:metal selectivity of at least 2:1 or above and a barrier layer:dielectric selectivity of at least 2:1 or above.

### EXAMPLES

The polishing experiments were carried out using the Mecapol 460 polisher produced by Steag. The polishing parameters are listed in Table 1. 200 mm wafers with coatings of Cu, TaN and SiO₂ were polished. The Cu layer was produced using 150 nm Cu seeds by sputtering and subsequent electroplating of 1000 nm of Cu. The TaN coating was deposited by sputtering a 90 nm TaN layer using a PVD (physical vapour deposition) process, while the SiO₂ was produced with a PVD process using TEOS. Cu and TaN removal rates were determined by measuring the resistance of the layers before and after polishing.

TABLE 1

| | |
|---|---|
| Polisher: | MECAPOL 460 |
| Working wheel | 45 rpm |
| (polishing pad) rotational | |
| speed | |
| Polishing head (wafer) | 30 rpm |
| rotational speed | |
| Applied pressure | 0.48 bar (7.0 psi) |
| Slurry flow rate | 180 ml/min |
| Polishing pad | Freudenberg FX9 on |
| | NPST 46 H |
| Back-surface pressure | 0 bar |

General procedure for production of the polishing slurries:

Polishing slurries with the solid contents and pH values indicated were produced from the silica sols indicated in the Examples by dilution with deionized water and addition of aqueous KOH (or of dilute sulphuric acid in the Comparative Examples). The wafers were polished immediately after the polishing slurries had been produced.

### Example 1

In this series of tests, polishing slurries were produced from a silica sol (Levasil® 50CK/30%-V1, Bayer AG) with a mean particle diameter of 80 nm and a solids content of 5% by weight of SiO₂. The pH was adjusted by adding aqueous KOH.

TABLE 2

| Concentration of SiO₂ | | Removal rate nm/min | | | Selectivities | | |
|---|---|---|---|---|---|---|---|
| [% by weight] | pH | Cu | TaN | SiO₂ | Cu | TaN | SiO₂ |
| 5 | 6 | 8 | 40 | 9 | 1 | 5 | 1.1 |
| 5 | 8 | 9 | 40 | 1 | 1 | 4.4 | 0.025 |
| 5 | 10 | 8 | 60 | 10 | 1 | 7.5 | 0.17 |

### Example 2

In this series of tests, polishing slurries were produced from a silica sol (Levasil® 100 K/30%-V1, Bayer AG) with a mean particle diameter of 30 nm and a solids concentration of 10% by weight of SiO₂. Then, aqueous KOH solutions were used to set different pHs of from 5-8, and stirring continued for one hour. The wafers were polished immediately after production of the polishing slurries. The removal rates and selectivities are listed in Table 3.

TABLE 3

| Polishing slurry | | Removal rate/nm/min | | | Selectivity | | |
|---|---|---|---|---|---|---|---|
| % by weight of SiO₂ | PH | Cu | TaN | SiO₂ | Cu | TaN | SiO₂ |
| 10 | 5 | 5 | 80 | 4 | 1 | 16 | 0.8 |
| 10 | 8 | 5 | 70 | 3 | 1 | 14 | 0.6 |

### Example 3

In this series of tests, polishing slurries were produced from a silica sol (Levasil® 100CK/30%-V1, Bayer AG) with a mean particle diameter of 30 nm and a solids content of 20% by weight of SiO₂. Then, aqueous KOH solutions were used to set various pHs of from 5-8, and stirring continued for one hour. The wafers were polished immediately after the polishing slurries had been produced. The removal rates and selectivities are listed in Table 4.

TABLE 4

| Polishing slurry | | Removal rate/Å/min | | | Selectivity | | |
|---|---|---|---|---|---|---|---|
| % by weight of SiO₂ | PH | Cu | TaN | SiO₂ | Cu | TaN | SiO₂ |
| 20 | 5 | 17 | 120 | 33 | 1 | 7 | 1.9 |
| 20 | 8 | 5 | 75 | 40 | 1 | 15 | 8 |

### Comparative Example 1

In this test, a polishing slurry was produced analogously to Example 1. The SiO₂ solids concentration was 5% by weight.

The pH was set to 2.2 using dilute H₂SO₄. The wafers were polished immediately after the polishing slurry had been produced. The removal rates and selectivities are listed in Table 5.

TABLE 5

| Removal rate/nm/min | | | Selectivity | | |
|---|---|---|---|---|---|
| Cu | TaN | Si O₂ | Cu | TaN | SiO₂ |
| 12 | 60 | 60 | 1 | 5 | 5 |

The Comparative Example shows that at low pHs, the required selectivities are not achieved, the amount of TaN and SiO₂ being removed being identical.

### Comparative Example 2

In this test, a polishing slurry was produced analogously to Example 2 with 10% by weight of SiO₂. Then, dilute H₂SO₄ was added in order to obtain a pH of 2. Stirring then continued for one hour. The wafers were polished immediately after the polishing slurry had been produced. The removal rates and selectivities are listed in Table 6.

TABLE 6

| Removal rate/Å/min | | | Selectivity | | |
|---|---|---|---|---|---|
| Cu | TaN | SiO₂ | Cu | TaN | SiO₂ |
| 10 | 70 | 230 | 1 | 7 | 23 |

It can be seen from the Comparative Example that the polishing slurry at a low pH of 2 does not have the selectivities found when using the polishing slurries according to the invention with higher pHs. The amount of SiO₂ removed is greater than the amount of TaN removed.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1.-5. (Cancelled)

6. A method of preparing a structure selected from the group consisting of metal structures and metal/dielectric structures, comprising:
(a) providing a composition comprising,
7 to 100% by volume of a cationically stabilized silica sol which contains 30% by weight of SiO₂ and the SiO₂ particles of which have a mean particle size of less than 300 nm, and
less than 0.05% by weight of oxidizing agent,
wherein said composition has a pH of from 4 to 10; and
(b) polishing said structure with said composition.

7. The method of claim 6, wherein the said structure comprises a metal selected from the group consisting of W, Al, Ru, Pt, Ir, Cu, Si, alloys thereof, carbides thereof and combinations thereof.

8. The method of claim 6, wherein the dielectric of said metal dielectric structure is selected from the group consisting of, polyimides, fluorinated polyimides, diamond-like carbons, polyarylethers, polyarylenes, parylene N, cyclotenes, polynorbonenes, polytetrafluoroethylene, silsesquioxanes, SiO₂ glass and combinations thereof.

9. A method of fabricating semiconductors, integrated circuits and microelectromechanical systems comprising providing dielectric structures prepared by the method of claim 6.

10. (Cancelled)
